(19) United States
(12) Patent Application Publication
(10) Pub. No.: US 2011/0244571 A1
(43) Pub. Date: Oct. 6, 2011

(54) CELL GROWTH

(75) Inventors: Haris Jamil, Libertyville, IL (US); James Hussey, Hawthorne Woods, IL (US); Nabil A. Amro, Prospect Heights, IL (US)

(73) Assignee: Nanolnk, Inc.

(21) Appl. No.: 13/122,557

(22) PCT Filed: Oct. 5, 2009
(86) PCT No.: PCT/US09/59566

§ 371 Date: Jun. 16, 2011

Related U.S. Application Data
(60) Provisional application No. 61/103,199, filed on Oct. 6, 2008.

(51) Int. Cl. C12N 5/071; C12N 5/0735; C12N 5/074; C12N 5/077; C12N 5/079; C12N 5/0781; C12N 5/0783; C12N 5/0793; C12N 5/0786; C12N 5/078; C12N 5/0787; B82Y 5/00

(52) U.S. Cl. 435/395; 977/700; 977/923; 977/888; 977/810; 977/832; 977/887

(57) ABSTRACT

Methods of preparing pre-engineered surfaces using various nanolithography techniques to generate, isolate, and multiply homogeneous cell populations. Surfaces can be treated by etching before exposure to biological systems like cells. Stem cell applications are described.

### PRIORITY CLAIM

This application claims priority to U.S. Provisional Application No. 61/103,199, filed Oct. 6, 2008, which is hereby incorporated by reference in its entirety.

### INTRODUCTION

Large amounts of time and effort have been devoted to the study of cells, the building blocks of life, including development of improved methods for growing and characterizing cells. An area of particular interest in the study of cells revolves around how cell behaviors, e.g. adhesion, growth, and differentiation, can be influenced by the cell environment, both in vivo and in vitro. For example, because of their ability to differentiate into many types of specialized cells, stem cells, and to a lesser but still significant degree, progenitor cells, have become the focus of research efforts that seek to understand how the ability of cells to differentiate can be influenced, and ultimately reliably controlled, such that pure cultures of desired “target” cells can be produced efficiently and inexpensively.

Various procedures designed to control cell differentiation have been described and are known in the art. For example, protocols have been published for differentiating embryonic stem cells and adult stem cells involving either addition of different growth factors, such as cytokines, hormones, ligands, or small molecular weight compounds in the culture medium known to induce stem cell differentiation, changing the topography of the extracellular matrix, or inserting specific genes or microRNA (for example, Goymer, P, 2008, Nature Rev Genetics 9, 251). However, known methods of cell differentiation typically provide insufficient yields of desired cells and unacceptable homogeneity of the final cell populations. Current protocols for in vitro differentiation generally give rise to heterogeneous populations of different cell lineages, with the desired cell type present only as a small proportion of the total number of cell types. As a consequence, there is a need for simple and reliable protocols for controlled differentiation of stem cells into pure, homogeneous cell populations, as well as for the isolation and multiplication of the populations obtained. Better cell growth control is needed, whether cells proliferate or are differentiated.

### SUMMARY

A variety of embodiments are described herein including, for example, articles, methods of making same, methods of using same, kits, and compositions. Pre-engineered surfaces for stem-cell differentiation are described herein with particular importance being attributed to surfaces comprising modified nanostructures.

Provided, for example, is a method of cell culture, comprising: (a) providing a substrate and a tip; (b) using the tip to apply a patterning compound to the substrate so as to produce a desired pattern which is a chemical etching resist; (c) etching the substrate chemically to produce a nanostructure; (d) modifying the nanostructure with a ligand to form a ligand-modified nanostructure; (e) contacting the ligand-modified nanostructure with at least one cell; (f) allowing the cell to grow; and (g) optionally, differentiating the cell.

Another embodiment provides a method of cell culture, comprising: (a) providing a substrate; (b) coating the substrate with a resist; (c) etching the resist with an electron beam to produce a patterned substrate; (d) evaporating metal on the patterned substrate; (e) removing the resist to produce a metal nanostructure, (f) modifying the metal nanostructure with a ligand;

(g) contacting the ligand-modified metal nanostructure with at least one cell; (h) allowing the cell to grow; and (i) optionally, differentiating the cell.

Another embodiment provides a method of cell culture, comprising: (a) providing a substrate; (b) coating the substrate with a resist; (c) etching the resist with an electron beam to produce a patterned substrate; (d) modifying the patterned substrate with a silane solution; (e) removing the resist to provide a silane-modified substrate; (f) contacting the silane-modified substrate with at least one cell; (g) allowing the cell to grow; and (h) optionally, differentiating the cell.

Another embodiment provides a method of cell culture, comprising: (a) providing a substrate; (b) coating the substrate with a metal; (c) coating the metal with a resist; (d) etching the resist with an electron beam to produce a patterned metal; (e) modifying the patterned metal with a silane solution; (f) removing the resist to provide a silane-modified metal; (g) contacting the silane-modified metal with at least one cell; (h) allowing the cell to grow; and (i) optionally, differentiating the cell.

Another embodiment provides a method of cell culture, comprising: (a) providing a substrate; (b) coating the substrate with a resist; (c) fabricating a resist nanostructure with a nanoimprint stamp; (d) using reactive ion etching to expose regions of the substrate; (e) evaporating a metal on the resist nanostructures and exposed substrate regions; (f) removing the resist to provide a metal nanostructure; (g) modifying the metal nanostructure with a ligand; (h) contacting the modified nanostructure with at least one cell; (i) allowing the cell to grow; and (j) optionally, differentiating the cell.

Another embodiment provides a method of cell culture, comprising: (a) providing a substrate; (b) coating the substrate with a resist; (c) fabricating a resist nanostructure with a nanoimprint stamp; (d) using reactive ion etching to expose regions of substrate; (e) exposing the exposed regions of substrate to a silane solution; (f) removing the resist to provide a silane nanostructure; (g) contacting the silane nanostructure with at least one cell; (h) allowing the cell to grow; and (i) optionally, differentiating the cell.

Another embodiment provides a method of cell culture, comprising: (a) providing a substrate; (b) coating the substrate with a metal; (c) coating the metal with a resist; (d) fabricating a resist nanostructure with a nanoimprint stamp; (e) using reactive ion etching to expose regions of the metal; (f) modifying the exposed metal regions with a ligand: (g) removing the resist to provide a ligand nanostructure; (h) contacting the ligand nanostructure with at least one cell; (i) allowing the cell to grow; and (j) optionally, differentiating the cell.

Another embodiment provides a method of cell culture, comprising: (a) providing a ligand-modified nanostructure prepared by a direct write nanolithography technique, wherein the direct write nanolithography technique is selected from the group consisting of dip pen nanolithography, microcontact printing, nanografting, nanopen reader writer nanolithography; (b) contacting the ligand-modified nanostructure with at least one cell; (c) allowing the cell to grow; and (d) optionally, differentiating the cell.

At least one advantage for at least one embodiment is improved versatility for the patterning.

At least one additional advantage for at least one embodiment is improved methods for commercial exploitation.

### BRIEF DESCRIPTION OF THE DRAWINGS

FIG. 1 shows a conventional DPN method for fabrication of nanostructures on a substrate.

FIG. 2 shows a scheme for DPN-based fabrication of homogeneous nanostructures on a substrate.

FIG. 3 shows schemes for EBL-based fabrication of homogeneous nanostructures on a substrate.

FIG. 4 shows a scheme for EBL-based fabrication of homogeneous nanostructures on a substrate.

FIG. 5 shows schemes for NIL-based fabrication of homogeneous nanostructures on a substrate.

FIG. 6 shows a scheme for NIL-based fabrication of homogeneous nanostructures on a substrate.

### DETAILED DESCRIPTION

The following references describe stem-cell growth, differentiation, and characterization.

UK provisional filing 0812789.6 filed Jul. 12, 2008; U.S. Provisional Application 61/099,182 filed Sep. 22, 2008, and PCT application PCT/IB2009/006521 filed Jul. 10, 2009 to Hunt et al.; are hereby incorporated by reference in their entireties including figures and working examples and claims. Also, U.S. provisional application No. 61/238,010 filed Aug. 28, to Amro et al., 2009 is incorporated by reference in its entirety.

Disclosed herein are methods and compositions that allow large surface fabrication of nanostructures comprising different molecules, wherein the large surface comprises homogeneous features with exact pitch across the surface, and wherein the surface is reusable. The present disclosure provides protocols for homogeneous compound deposition, fast processing, stable structures, and reusable substrates with ligands of interest. Such protocols may benefit, for example, stem-cell differentiation include, among others: homogeneous stem cell differentiation; reliable procedures, manufacturability, and high throughput processes.

All technical terms used herein are terms commonly used in cell biology, biochemistry, molecular biology, and nanolithography and can be understood by one of ordinary skill in the art to which this invention belongs. These technical terms can be found in the current editions of Molecular Cloning: A Laboratory Manual, (Sambrook et al., Cold Spring Harbor); Gene Transfer Vectors for Mammalian Cells (Miller & Calos eds.); and Current Protocols in Molecular Biology (F. M. Ausubel et al. eds., Wiley & Sons). Cell biology, protein chemistry, and antibody techniques can be found in Current Protocols in Protein Science (J. E. Colligan et al. eds., Wiley & Sons); Current Protocols in Cell Biology (J. S. Bonifacino et al., Wiley & Sons) and Current Protocols in Immunology (J. E. Colligan et al. eds., Wiley & Sons). Reagents, cloning vectors, and kits for genetic manipulation referred to in this disclosure are available from commercial vendors such as BioRad, Stratagene, Invitrogen, ClonTech, and Sigma-Aldrich Co.

Cell culture methods are described generally in the current edition of Culture of Animal Cells: A Manual of Basic Technique (R. I. Freshney ed., Wiley & Sons); General Techniques of Cell Culture (M. A. Harrison & I. F. Rae, Cambridge Univ. Press), and Embryonic Stem Cells: Methods and Protocols (K. Turksen ed., Humana Press). Other texts include Creating a High Performance Culture (Aroselli, Hu. Res. Dev. Pr. 1996) and Limits to Growth (D. H. Meadows et al., Universe Publ. 1974). Tissue culture supplies and reagents are available from commercial vendors such as Gibco/BRL, Nalgene-Nunc International, Sigma Chemical Co., and ICN Biomedicals.

Nanolithography methods, such as direct-write technologies can be carried out by methods described in, for example, Direct-Write Technologies for Rapid Prototyping Applications: Sensors, Electronics, and Integrated Power Sources, Ed. by A. Pique and D. B. Chrisey, Academic Press, 2002. Chapter 10 by Mirkin, Demers, and Hong, for example, describes nanolithographic printing at the sub-100 nanometer length scale, and is hereby incorporated by reference (pages 303-312). Pages 311-312 provide additional references on scanning probe lithography and direct-write methods using patterning compounds delivered to substrates from nanoscopic tips which can guide one skilled in the art in the practice of the present invention. Nanolithography and nanofabrication is also described in Marc J. Madou's Fundamentals of Microfabrication, The Science of Miniaturization, 2.sup.nd Ed., including metal deposition at pages 344-357. See also, e.g., U.S. Pat. Nos. 6,827,979; 6,635,311; and 6,867,443.

Although this specification provides guidance to one skilled in the art, including reference to technical literature, mere reference does not constitute an admission that the technical literature is prior art.

### A. Patterning and Nanolithography

A variety of patterning and nanolithography methods can be used. For example, DPN printing is one example. Dip Pen Nanolithography (“DPN”) printing and deposition methods are extensively described in the following patent applications and patent publications, which are hereby incorporated by reference in their entirety and support the disclosure for the present inventions. No admission is made that any of these references are prior art.
1. U.S. Provisional application 60/115,133 filed Jan. 7, 1999 (“Dip Pen Nano lithography”).
2. U.S. Provisional application 60/157,633 filed Oct. 4, 1999 (“Methods Utilizing Scanning Probe Microscope Tips and Products Therefor or Produced Thereby”).
3. U.S. Regular patent application Ser. No. 09/477,997 filed Jan. 5, 2000 (“Methods Utilizing Scanning Probe Microscope Tips and Products Therefor or Produced Thereby”), now U.S. Pat. No. 6,635,311 to Mirkin et al. issued Oct. 21, 2003.
4. U.S. Provisional application 60/207,713 filed May 26, 2000 (“Methods Utilizing Scanning Probe Microscope Tips and Products Therefor or Produced Thereby”). This application, for example, describes wet chemical etching, working examples, references, and figures, which are all incorporated by reference in their entirety.
5. U.S. Provisional application 60/207,711 filed May 26, 2000 (“Methods Utilizing Scanning Probe Microscope Tips and Products Therefor or Produced Thereby”).
6. U.S. Regular application Ser. No. 09/866,533 filed May 24, 2001 (“Methods Utilizing Scanning Probe Microscope Tips and Products Therefor or Produced Thereby”). This application, for example, describes wet chemical etching, working examples (e.g., example 5), references, and figures, which are all incorporated by reference in their entirety.
7. U.S. patent publication number 2002/0063212 A1 published May 30, 2002 (“Methods Utilizing Scanning Probe Microscope Tips and Products Therefor or Produced Thereby”).
8. U.S. patent publication number 2002/0122873 A1 published Sep. 5, 2002 (“Nanolithography Methods and Products Produced Therefor and Produced Thereby”).
9. PCT publication number WO 00/41213 A1 published Jul. 13, 2000 based on PCT application no. PCT/US00/00319 filed Jan. 7, 2000 (“Methods Utilizing Scanning Probe Microscope Tips and Products Therefor or Produced Thereby”).
10. PCT publication number WO 01/91855 A1 published Dec. 6, 2001 based on PCT application no. PCT/US01/17067 filed May 25, 2001 (“Methods Utilizing Scanning Probe Microscope Tips and Products Therefor or Produced Thereby”).
11. U.S. Provisional application 60/326,767 filed Oct. 2, 2001, (“Protein Arrays with Nanoscopic Features Generated by Dip-Pen Nanolithography”), now published 2003/0068446 on Apr. 10, 2003 to Mirkin et al.
12. U.S. Provisional application 60/337,598 filed Nov. 30, 2001, (“Patterning of Nucleic Acids by Dip-Pen Nanolithography”) and U.S. regular application Ser. No. 10/307,515 filed. Dec. 2, 2002 to Mirkin et al.
13. U.S. Provisional application 60/341,614 filed Dec. 17, 2001, (“Patterning of Solid State Features by Dip-Pen Nanolithography”), now published 2003/0162004 Aug. 28, 2003 to Mirkin et al.
14. U.S. Provisional application 60/367,514 filed Mar. 27, 2002, (“Method and Apparatus for Aligning Patterns on a Substrate”) and publication no. 2003/0185967 on Oct. 2, 2003 to Eby et al.
15. U.S. Provisional application 60/379,755 filed May 14, 2002, (“Nanolithographic Calibration Methods”) and U.S. regular application Ser. No. 10/375,060 filed Feb. 28, 2003 to Cruchon-Dupeyrat et al.

In general, state of the art DPN printing and deposition-related products, including hardware, software, and instrumentation are also available from NanoInk, Inc. (Chicago, Ill.), and these can be used in practicing the methods discloses herein.

Other methods and articles are described in Huo et al., Science, 321, 5896, 1658-1660, including methods sometimes called “polymer pen lithography.”

Parallel methods of the DPN printing process can be carried out as described in, for example, U.S. Pat. No. 6,642,129 to Liu et al. issued Nov. 4, 2003.

In addition, the following papers describes wet chemical etching procedures used in conjunction with direct-write nanolithography, and are hereby incorporated by reference in its entirety including figures, references, and working examples: Zhang et al., “Dip-Pen Nanolithography-Based Methodology for Preparing Arrays of Nanostructures Functionalized with Oligonucleotides”; Adv. Mat., 2002, 14, No. 20, Oct. 16, pages 1472-1474; Zhang et al., “Biofunctionalized Nanoarrays of Inorganic Structures Prepared by Dip-Pen Nanolithography”; Nanotechnology, 2003, 14, 1113-1117.

The text Fundamentals of Microfabrication, The Science of Minitaturization, 2^{nd} Ed., Marc J. Madou, describes micro and nanotechnologies including additive and substractive methods, for example, lithography, (Chapter 1), pattern transfer with dry etching methods (Chapter 2), pattern transfer with additive methods (Chapter 3), wet bulk micromachining (Chapter 4).

The text Direct-Write Technologies for Rapid Prototyping Applications: Sensors, Electronics, and Integrated Power Sources (Eds. A. Pique and D. B. Chrisey), describes micro and nanotechnologies including additive and substractive methods. For example, bulk micromachining and etching are described on pages 617-619. DPN printing on the Sub-100 nanometer length scale is described in Chapter 10.

Self-assembled monolayers, etching, and microfabrication are further described in, for example, U.S. Pat. Nos. 5,618,760 to Soh et al; 5,620,850 to Bamdad et al.; and 5,512,131 to Kumar et al.

Additional references include, for example, US provisional filings filed Jan. 26, 2009 Ser. Nos. 61/147,448; 61/147,449; 61/147,451; and 61/147,452, to Amro et al., including applications for cell and stem cell growth and culturing, including coating of tips, heating of substrates, leveling methods, and cell growth studies for homogeneous patterning.

Methods for producing DPN-based surfaces are known in the art and described in the references cited above. DPN printing can be used in conjunction with etching in practicing methods of the present application, whether wet or dry. In particular, a tip such as a nanoscopic tip or an SPM tip can be used to deliver a patterning compound to a substrate of interest in a pattern of interest, all as described above, and the patterning compound functions as an etching resist in one or more subsequent etching procedures. The patterning compounds can be used to pattern the substrate prior to any etching or after one or more etching steps have been performed to protect areas exposed by the etching step(s).

### Electron Beam Nanolithography (“EBL”)

EBL is a high-resolution technique for patterning involving high-energy electrons focused into a beam that can be used to expose electron-sensitive resists. EBL is described in detail in many references, such as, for example: SPIE Handbook of Microlithography, Micromachining and Microfabrication, Chapter 2, McCord, M. A.; M. J. Rooks (2000); Wallraff and Hinsberg, Chem. Rev., 99:1801 (1999); and Xia et al., Chem. Rev., 99:1823 (1999).

EBL can be used to form either additive or subtractive patterns by methods known in the art. Any EBL resist with appropriate sensitivity, tone, resolution, and etching resistance can be used in practicing methods of the present application. Both positive and negative resists may be used in practicing methods disclosed herein.

Electron Beam Lithography (“EBL”) techniques are described in SPIE Handbook of Microlithography, Micromachining and Microfabrication, Chapter 2, McCord, M. A.; M. J. Rooks (2000); Wallraff and Hinsberg, Chem. Rev., 99:1801 (1999); and Xia et al., Chem. Rev., 99:1823 (1999).

### Nanoimprint Lithography (“NIL”)

NIL is a technique based on pressing a mold into a resist coated on a substrate to create a relief pattern followed by removing the compressed material. NIL techniques are known to those skilled in the art, and methods and materials are described in, for example, Chou et al., “Imprint Lithography with 25-Nanometer Resolution,” Science 5 Apr. 1996, Vol. 272. no. 5258, pp. 85-87; “Nanoimprint Lithography” by S. Y. Chou et al., J. Vac. Sci. Technol. B, 14(6), November/December 1996, pages 4129-4133; U.S. Pat. No. 7,128,559 to Gregory et al., U.S. Pat. No. 5,772,905 to Chou, and U.S. Pat. No. 6,309,580 to Chou et al.

Any suitable nanoimprint stamp and resist known in the art may be used in practicing methods of the present application. Removal of compressed material can be accomplished by any method known to one skilled in the art, e.g. by reactive ion etching.

Nanoimprint Lithography (“NIL”) techniques are described in Chou et al., “Imprint Lithography with 25-Nanometer Resolution,” Science 5 Apr. 1996, Vol. 272. no. 5258, pp. 85-87; “Nanoimprint Lithography” by S. Y. Chou et al., J. Vac. Sci. Technol. B, 14(6), November/December 1996, pages 4129-4133; U.S. Pat. No. 7,128,559 to Gregory et al., U.S. Pat. No. 5,772,905 to Chou, and U.S. Pat. No. 6,309,580 to Chou et al.

### Substrates

Substrates suitable for use in nanolithography techniques are known in the art. For example, see U.S. Pat. No. 7,291,284 to Mirkin et al. and U.S. Pat. No. 6,635,311 to Mirkin et al., the contents of which are hereby incorporated by reference in their entirety.

Substrates suitable for use in nanolithography techniques disclosed herein can include any suitable nanolithography substrate. For example, the substrate can include a semiconductor, a combination of semiconductors, a metal such as, for example, gold, silver, copper, or palladium, a metal oxide or combinations of metal oxides, a superconducting material, magnetic material, silicon, silicon oxides, polymers, or coated polymers. The substrate can comprise multiple layers. Early steps can remove the top layers of the substrate, and later steps can remove lower layers of the substrate. The layers can be conductive, semiconductive, or insulating layers. The layers can be hard inorganic materials or soft organic materials, or combinations thereof. In preferred embodiments, the layers comprise conductive layer over a semiconductive layer. The semiconductor can be in an undoped or doped form. A variety of semiconductor materials can be used including, for example, II-VI and III-V types. A preferred example is silicon.

### Tips

Tips suitable for use in nanolithography techniques are known in the art. See, for example, U.S. Pat. No. 6,635,311 to Mirkin et al. In particular, a tip such as a nanoscopic tip or an SPM tip can be used to deliver a patterning compound to a substrate of interest in a pattern of interest. For direct write lithography steps, the tips can be hollow or non-hollow, and the ink can be supplied in a continuous or a non-continuous manner as described in references listed above. Devices including a single tip or multiple tips can be used to practice embodiments disclosed herein.

### Patterning Inks, Compositions, and Compounds

Many suitable patterning inks and compounds are known in the art. See, for example, patterning compounds described in U.S. Pat. No. 6,635,311 to Mirkin et al., and references cited therein describing patterning compounds.

A patterning ink or compound may be supplied to the tip in a continuous or non-continuous manner and can commonly chemisorb or covalently bond to the substrate. The patterning compound may comprise a sulfur-containing compound, nitrogen-containing compound, or silicon-containing compound. The patterning compound can commonly comprise a compound capable of forming a self assembled monolayer.

### Patterns

Suitable patterns for patterning compounds on the substrate are known in the art and are described, for example, in U.S. patent application Ser. No. 10/261,663 to Mirkin et al., and U.S. Pat. No. 6,635,311 to Mirkin et al.

Patterns may comprise dots, lines, or combinations of dots and lines. Lines can commonly have a width of about 15 nm to about 250 nm. Dots may be formed in any suitable geometry, such as, for example, circle, oval, square, rectangle, triangle, or star, and commonly have individual diameters ranging from about, for example, 10 nm to about 200 nm. The pitch of nanostructures in the pattern can be if desired constant across the substrate, and commonly can be, for example, about 75 nm to about 2000 nm.

### B. Ligands and Surface Modifying Agents

Ligands for modifying nanostructures disclosed herein are described in U.S. Patent Application “Materials and Methods for Cell Growth” filed Sep. 22, 2008, which is hereby incorporated by reference in its entirety.

Suitable ligands for embodiments disclosed herein typically comprise biologically active functional groups on one location of the ligand, and a functional group capable of binding with a nanostructure on a different location of the ligand such that the biologically active functional group is available for binding to a biological entity, e.g. a cell, when the functional group capable of binding with the nanostructure is bound to the nanostructure. Biologically active functional groups can include: ethyl groups; isopropyl groups; cyclohexyl groups; aryl groups; allyl groups; alkynyl groups; hydroxyl (alcohol) groups; ether groups; morpolino groups; ethylene glycosylated groups; polyethylene glycosylated groups; simple sugars, such as glucose, ribose, heparose, or mannose; carboxylate groups; sulfate groups; phosphate groups; phenoxide groups; amino groups; dialkylamino groups; alkylamino groups; phosphine groups; and amino acids. Functional groups capable of binding with a nanostructure can include, for example, functional groups that include sulfur, such as, for example, thiols, functional groups that include nitrogen, such as, for example, amines, or functional groups that include silanes. Other examples known in the art can be used.

In some embodiments, ligands can comprise growth factors, cytokines, inhibitors of gene regulation, activators of gene regulation, growth hormones, peptides, or receptors. Other examples known in the art can be used.

In other embodiments, exemplary ligands may be selected from among the non-exhaustive list presented in Table 1 below.

TABLE 1
Exemplary Ligands

| | | | mp |
|---|---|---|---|
| Chemical Name | Formula | Abbreviation | (° C.) |
| Mercaptoundecanol | HSC₁₁OH | MUD | 33-37 |
| Mercaptohexadecanoic | HSC₁₅COOH | MHA | 65-69 |
| acid | | | |
| Hexadecane thiol | HSC₁₅CH₃ | HDT | 20-24 |
| Octadecane thiol | HSC₁₅CH₃ | ODT | 30-33 |
| 11-mercaptoundecyl | HSC₁₁NH₂ | MUA | 134-137 |
| amine | | | |
| 2-{2-{2-(1- | HSC₁₁-PEG₃ | HS-PEG3 | 182-186 |
| mercaptoundec- | | | |
| 11-yloxy)-ethoxy]- | | | |
| ethoxy}-ethanol | | | |
| Thiotic acid-2-{2- | (HS—CH₂—)₂C₁₁- | (HS—CH2—)2- | ?? |
| [2-(1-mercaptoundec- | PEG₃ | PEG3 | |
| 11-yloxy)-ethoxy]- | | | |
| ethoxy}-ethanol | | | |

### C. Cells and Cell Growth

Cells used for growth, proliferation, and differentiation studies and methods and materials for culturing those cells are known in the art. See, for example, U.S. Pat. No. 7,374,934 to Keller et al., U.S. Pat. No. 5,453,357 to Hogan, U.S. Pat. No. 5,166,065 to Williams et al., and U.S. patent application “Materials and Methods for Cell Growth” filed Sep. 22, 2008.

Cells used in the present disclosure may include, for example, stem cells and progenitor cells. The stem cells and progenitor cells can be of any origin, e.g., mammalian, avian, etc. Commonly, the stem cells and progenitor cells may be of human origin. The stem cells can be adult stem cells or embryonic stem cells. The progenitor cells can originate from any tissue in which they reside.

Stem cells are cells found in most, if not all, multi-cellular organisms. They are characterized by the ability to renew themselves through mitotic cell division and differentiating into a diverse range of specialized cell types. The two broad types of mammalian stem cells are: embryonic stem cells that are isolated from the inner cell mass of blastocysts, and adult stem cells that are found in adult tissues. In a developing embryo, stem cells can differentiate into all of the specialized embryonic tissues. In adult organisms, stem cells and progenitor cells act as a repair system for the body, replenishing specialized cells, but also maintain the normal turnover of regenerative organs, such as blood, skin or intestinal tissues.

Cells, growth of cells, cell differentiation, and stem cells are generally known in the art. See, for example, Essentials of Stem Cell Biology, (Ed. R. Lanza), 2006; Cell Biology, 2″ Ed., Pollard and Earnshaw, 2008; Gilbert, Developmental Biology, 5^{th} ed., 1997; Cell Lineage Specification and Patterning of the Embryo, (ed. Etkin, Jeon), 2001. See Curran et al., Biomaterials, 27 (2006), 4783-4793; Curran et al., Biomaterials 26 (2005), 7057-7067.

Suitable source cells for culturing stem cells include established lines of pluripotent cells derived from tissue formed after gestation. Exemplary cells include mesenchymal stem cells, which can be obtained by methods known in the art. For example, and in no way limiting the invention, mesenchymal stem cells can be obtained from raw, unpurified bone marrow or ficoll-purified bone marrow monocytes plated directly into cell culture plates or flasks. Exemplary cells include those disclosed in Curran et al. Biomaterials 27 (2006), 4783-4793.

Without limitation, cells can include various adult human stem cells, e.g. mesenchymal cells, hematopoietic cells, neural stem cells, epithelial cells, and skin cells. Non-terminally differentiated cells can be used.

Various methods may be used for cell growth and differentiation, which may be used for screening a population of cells for selecting a differentiated cell. In no way limiting, a variety of molecular and/or cell biology techniques may be used to assess cell differentiation. For example, RT-PCR may be used to detect a specific genetic marker indicative of cell differentiation. Likewise, a western blot and/or Bradford protein assay may be performed to analyze protein expression commensurate with a specific cell type. Similarly, various cell sorting techniques such as fluorescence-activated cell sorting (FACS) may used to determine cell differentiation.

For example and in no way limiting, populations of the characterized stem cells can be seeded onto materials disclosed herein, commonly at a seeding density of about 10³ to 10⁴ cells/ml (total). The cells can be cultured on the materials in the presence of commercially-defined basal medium for any suitable amount of time, e.g., 24 hours. The cultured cells can be analyzed for cell adhesion, focal contact formation, formation of cytoskeletal components and overall morphology by methods known in the art. Longer studies for, for example, 14 and/or 28 days can be conducted by methods known in the art.

### D. Cell Differentiation

If desired, cells can be differentiated. Cell culture methods disclosed herein can produce homogeneous cultures of differentiated cells. The homogeneous cultures of differentiated cells include but are not limited to chondrogenic cells, osteogenic cells, neurogenic cells, myogenic cells, or adipogenic cells, blood cells e.g., red blood cells, B lymphocytes, T lymphocytes, natural killer cells, neutrophils, basophils, eosinophils, monocytes, macrophages and platelets, brain cells, e.g. neurons, oligodendrocytes, and astrocytes, absorptive cells, globlet cells, paneth cells, enteroendocrine cells, keratinocytes, cardiac muscle cells, skeletal muscle cells or liver cells. Commonly, differentiated cells can be greater than, for example, 60%, or 70%, or 80% homogeneous.

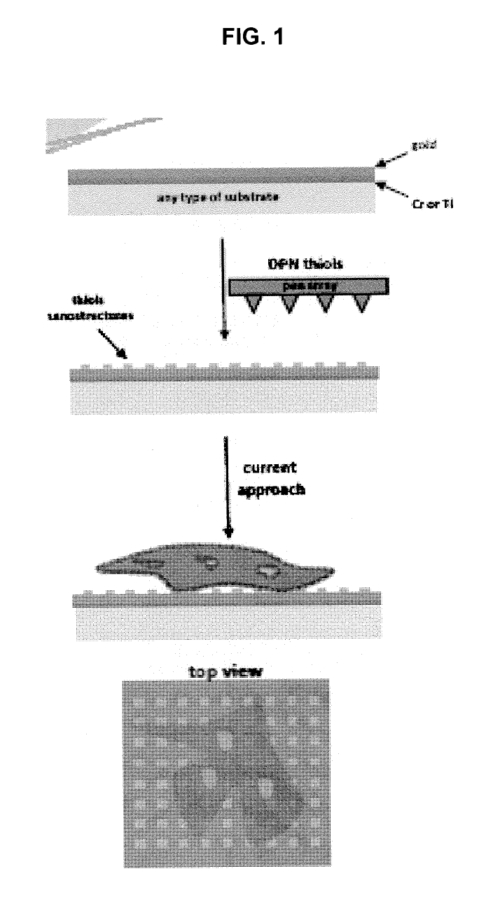

One embodiment showing current approaches is shown in FIG. 1.

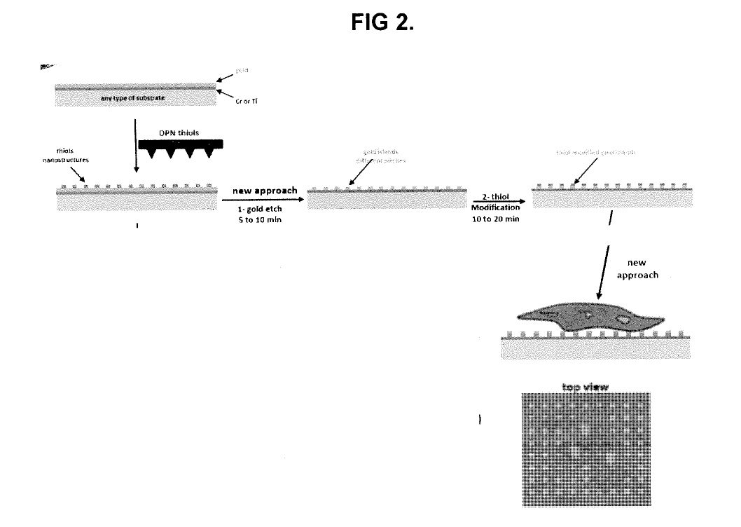

One embodiment, shown schematically in FIG. 2, provides a method of cell culture utilizing a reusable, pre-engineered surface prepared using DPN technology that involves the steps of providing a substrate and a tip, using the tip to apply a patterning compound to the substrate so as to produce a desired pattern which is a chemical etching resist, etching the substrate chemically to produce a nanostructure, modifying the nanostructure with a ligand to form a ligand-modified nanostructure; contacting the ligand-modified nanostructure with at least one cell; allowing the cell to grow; and differentiating the cell. Examples of materials and methods required to produce the pre-engineered surface are described in DPN-related references above. Optionally the substrate comprises a metal surface. Optionally the substrate comprises gold. Optionally the tip is a scanning probe microscope tip. Optionally the patterning compound can chemisorb or covalently bond to the substrate. Optionally the patterning compound is a sulfur-containing compound. Optionally the patterning compound is a biologically active compound. Optionally the desired pattern comprises a self-assembled monolayer. Optionally the desired pattern has a pitch adapted to provide effective results. Optionally the nanostructure formed after etching has a diameter adapted to provide effective results. Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an adult stem cell. Optionally the at least one cell comprises an adult mesenchymal, hematopoietic, neural, epithelial, or skin stem cell. Optionally the at least one cell comprises a progenitor cell. Optionally the differentiated cell is a chondrogenic cell, an osteogenic cell, a neurogenic cell, a myogenic cell, and an adipogenic cell. Optionally the differentiated cell is a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

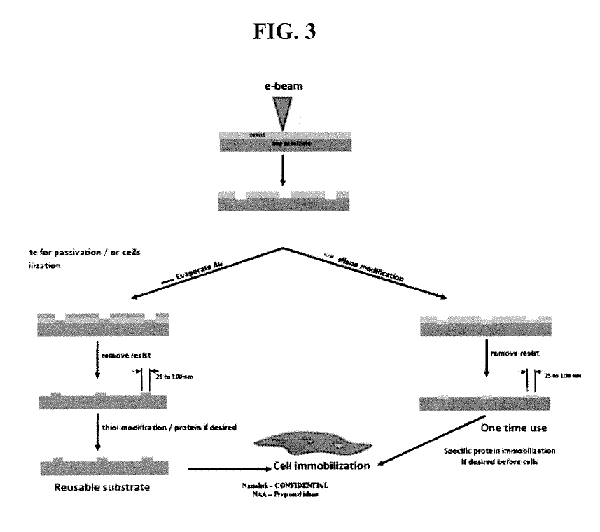

Another embodiment, shown schematically in FIG. 3, provides a method of cell culture utilizing a reusable, pre-engineered surface prepared using EBL technology that involves providing a substrate; coating the substrate with a resist; etching the resist with an electron beam to produce a patterned substrate; evaporating metal on the patterned substrate; removing the resist to produce a metal nanostructure; modifying the metal nanostructure with a ligand; contacting the ligand-modified metal nanostructure with at least one cell; allowing the cell to grow; and differentiating the cell. Examples of materials and methods used to produce the pre-engineered surface are described in EBL-related references above. Optionally the substrate comprises a metal. Optionally the substrate comprises a semiconductor. Optionally the resist comprises a positive resist. Optionally the patterned substrate has a pitch adapted to provide effective results. Optionally the evaporated metal comprises, for example, gold. Optionally removing the resist can be carried out by known methods. Optionally the metal nanostructure has a pitch adapted to provide effective results. Optionally the metal nanostructures are circular, oval, square, rectangular, or star shaped. Optionally metal nanostructure has a diameter adapted to provide effective results. Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an embryonic stem cell. Optionally the at least one cell comprises an adult stem cell. Optionally the at least one cell comprises an adult mesenchymal, hematopoietic, neural, epithelial, or skin stem cell. Optionally the at least one cell comprises a progenitor cell. Optionally allowing the cell to grow further comprises steps known in the art. Optionally the differentiated cell is a chondrogenic cell, an osteogenic cell, a neurogenic cell, a myogenic cell, and an adipogenic cell. Optionally the differentiated cell is a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

Another embodiment, shown schematically in FIG. 3, provides a method of cell culture utilizing a pre-engineered surface prepared using EBL technology that involves providing a substrate; coating the substrate with a resist; etching the resist with an electron beam to produce a patterned substrate; modifying the patterned substrate with a silane solution; removing the resist to provide a silane-modified substrate; contacting the silane-modified substrate with at least one cell; allowing the cell to grow; and differentiating the cell. Examples of materials and methods used to produce the pre-engineered surface are described in EBL-related references above. Optionally the substrate comprises a polymer. Optionally the substrate comprises silicon. Optionally the resist comprises a negative resist. Optionally the patterned substrate has a pitch adapted to provide effective results. Optionally the silane solution comprises 0.5-2% (w/w) n-octadecyltrimethoxysilane (OTS)/toluene solution. Optionally removing the resist comprises materials known in the art. Optionally the silane-modified substrate has a pitch adapted to provide effective results. Optionally the silane-modified substrate regions are circular, oval, square, rectangular, or star shaped. Optionally the silane-modified substrate regions have a diameter adapted to provide effective results. Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an embryonic stem cell. Optionally the at least one cell comprises an adult stem cell. Optionally the at least one cell comprises an adult mesenchymal, hematopoietic, neural, epithelial, or skin stem cell. Optionally the at least one cell comprises a progenitor cell. Optionally allowing the cell to grow further comprises methods known in the art. Optionally the differentiated cell is a chondrogenic cell, an osteogenic cell, a neurogenic cell, a myogenic cell, and an adipogenic cell. Optionally the differentiated cell is a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

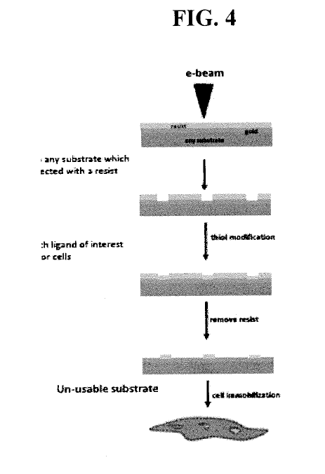

Another embodiment, shown schematically in FIG. 4, provides a method of cell culture utilizing a pre-engineered surface prepared using EBL technology that involves providing a substrate, coating the substrate with a metal, coating the metal with a resist, etching the resist with an electron beam to produce a patterned metal, modifying the patterned metal with a silane solution, removing the resist to provide a silane-modified metal, contacting the silane-modified metal with at least one cell, allowing the cell to grow, and differentiating the cell. Examples of materials and methods used to produce the pre-engineered surface are described in EBL-related references above. Optionally the substrate comprises a polymer. Optionally the metal comprises, for example, gold. Optionally the resist comprises a positive resist. Optionally the patterned metal has a pitch adapted to provide effective results. Optionally the silane solution comprises 0.5-2% (w/w) n-octadecyltrimethoxysilane (OTS)/toluene solution. Optionally removing the resist comprises materials known in the art. Optionally the silane-modified metal regions have a pitch adapted to provide effective results. Optionally the silane-modified metal region is circular, oval, square, rectangular, or star shaped. Optionally the silane-modified metal region has a diameter adapted to provide effective results. Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an embryonic stem cell. Optionally the at least one cell comprises an adult stem cell. Optionally the at least one cell comprises an adult mesenchymal, hematopoietic, neural, epithelial, or skin stem cell. Optionally the at least one cell comprises a progenitor cell. Optionally allowing the cell to grow further comprises steps known in the art. Optionally the differentiated at least one cell is a chondrogenic cell, an osteogenic cell, a neurogenic cell, a myogenic cell, and an adipogenic cell. Optionally the differentiated at least one cell a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

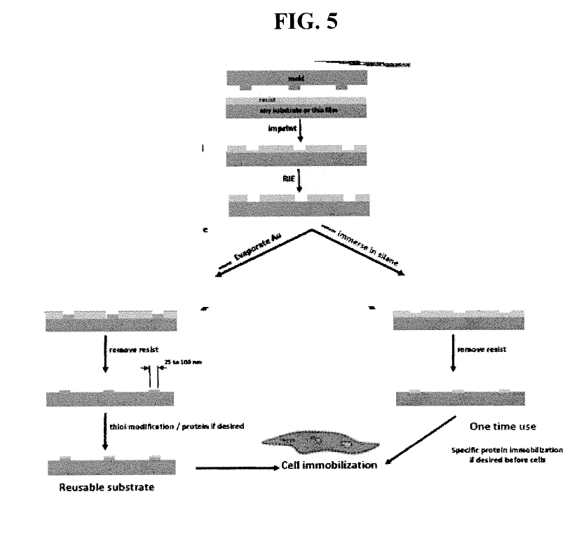

Another embodiment, shown schematically in FIG. 5, provides a method of cell culture, utilizing a reusable pre-engineered surface prepared using NIL technology that involves providing a substrate, coating the substrate with a resist, fabricating a resist nanostructure with a nanoimprint stamp; using reactive ion etching to expose regions of the substrate, evaporating a metal on the resist nanostructures and exposed substrate regions, removing the resist to provide a metal nanostructure, modifying the metal nanostructure with a ligand, contacting the modified nanostructure with at least one cell; allowing the cell to grow; and differentiating the cell. Examples of materials and methods required to produce the pre-engineered surface are described in NIL-related references above. Optionally the substrate comprises a polymer. Optionally the substrate comprises silicon. Optionally the resist comprises a negative resist. Optionally the resist nanostructure has a pitch adapted to provide effective results. Optionally the metal comprises, for example, gold. Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an embryonic stem cell. Optionally the at least one cell comprises an adult stem cell. Optionally the at least one cell comprises an adult mesenchymal, hematopoietic, neural, epithelial, or skin stem cell. Optionally the at least one cell comprises a progenitor cell. Optionally the differentiated at least one cell is a chondrogenic cell, an osteogenic cell, a neurogenic cell, a myogenic cell, and an adipogenic cell. Optionally the differentiated at least one cell is a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

Another embodiment, shown schematically in FIG. 5, provides a method of cell culture utilizing a pre-engineered surface prepared using NIL technology that involves providing a substrate, coating the substrate with a resist, fabricating a resist nanostructure with a nanoimprint stamp, using reactive ion etching to expose regions of substrate, exposing the exposed regions of substrate to a silane solution, removing the resist to provide a silane nanostructure, contacting the silane nanostructure with at least one cell, allowing the cell to grow; and differentiating the cell. Examples of materials and methods required to produce the pre-engineered surface are described in NIL-related references above. Optionally the substrate comprises a polymer. Optionally the substrate comprises silicon. Optionally the resist comprises a positive resist. Optionally the silane solution comprises 0.5-2% (w/w) n-octadecyltrimethoxysilane (OTS)/toluene solution. Optionally the silane nanostructure is circular, oval, square, rectangular, or star shaped.

Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an embryonic stem cell. Optionally the at least one cell comprises an adult stem cell. Optionally the at least one cell comprises an adult mesenchymal, hematopoietic, neural, epithelial, or skin stem cell. Optionally the differentiated cell is a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

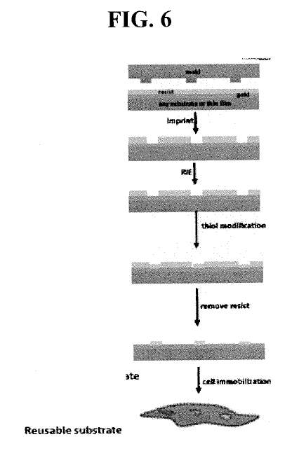

Another embodiment, shown schematically in FIG. 6, provides a method of cell culture utilizing a reusable pre-engineered surface prepared using NIL technology that involves providing a substrate, coating the substrate with a metal, coating the metal with a resist, fabricating a resist nanostructure with a nanoimprint stamp, using reactive ion etching to expose regions of the metal, modifying the exposed metal regions with a ligand, removing the resist to provide a ligand nanostructure, contacting the ligand nanostructure with at least one cell, allowing the cell to grow; and differentiating the cell. Examples of materials and methods required to produce the pre-engineered surface are described in NIL-related references above. Optionally the substrate comprises a polymer. Optionally the resist comprises a positive resist. Optionally the exposed regions of metal are circular, oval, square, rectangular, or star shaped. Optionally the ligand is a sulfur-containing compound. Optionally the ligand is a growth factor, cytokine, inhibitor of gene regulation, activator of gene regulation, growth hormone, peptide, or receptor. Optionally the at least one cell comprises a stem cell. Optionally the at least one cell comprises an embryonic stem cell. Optionally the at least one cell comprises an adult stem cell. Optionally the at least one cell comprises an adult mesenchymal, hematopoietic, neural, epithelial, or skin stem cell. Optionally the at least one cell comprises a progenitor cell. Optionally the differentiated cell is a chondrogenic cell, an osteogenic cell, a neurogenic cell, a myogenic cell, and an adipogenic cell. Optionally the differentiated cell is a red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, and keratinocyte.

Another embodiment of the present application provides a method of cell culture utilizing a reusable, pre-engineered surface prepared using nanolithography technology involving providing a ligand-modified nanostructure prepared by a direct write nanolithography technique, wherein the direct write nanolithography technique is dip pen nanolithography, microcontact printing, nanografting, or nanopen reader writer nanolithography; contacting the ligand-modified nanostructure with at least one cell; allowing the cell to grow; and differentiating the cell.

Examples of materials and methods required to produce the pre-engineered surface are described in direct write nanolithography-related references cited above.

Priority to U.S. Provisional Application No. 61/103,199, filed Oct. 6, 2008, is hereby incorporated by reference in its entirety including applications.

What is claimed is:

1. A method of cell culture, comprising:
a) providing a substrate and a tip;
b) using the tip to apply a patterning compound to the substrate so as to produce a desired pattern which is a chemical etching resist;
c) etching the substrate chemically to produce a nanostructure;
d) modifying the nanostructure with a ligand to form a ligand-modified nanostructure;
e) contacting the ligand-modified nanostructure with at least one cell;
f) allowing the cell to grow; and
g) optionally, differentiating the cell.

2. The method of claim 1, wherein the substrate comprises a metal surface.

3. The method of claim 1, wherein the tip is a scanning probe microscope tip.

4. The method of claim 1, wherein the patterning compound can chemisorb or covalently bond to the substrate.

5. The method of claim 1, wherein the patterning compound is a sulfur-containing compound or biologically active compound.

6. The method of claim 1, wherein the desired pattern comprises a self-assembled monolayer.

7. The method of claim 1, wherein the desired pattern has a pitch of about 10 nm to about 2,000 nm.

8. The method of claim 1, wherein the nanostructure formed after etching has a diameter of about 10 nm to about 2,000 nm.

9. The method of claim 1, wherein the ligand is a sulfur-containing compound, growth factor, cytokine, inhibitor of gene regulation, activator of gene regulation, growth hormone, peptide, or receptor.

10. The method of claim 1, wherein allowing the at least one cell to grow produces an expanded homogenous cell population.

11. The method of claim 1, wherein the cell is differentiated, and the differentiated at least one cell is a stem cell, embryonic stem cell, adult stem cell, adult mesenchymal, hematopoietic, neural, epithelial, skin stem cell, progenitor cell.

12. The method of claim 1, wherein the cell is differentiated, and the differentiated at least one cell is a chondrogenic cell, an osteogenic cell, a neurogenic cell, a myogenic cell, adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocyte.

13. A method of cell culture, comprising:
a) providing a substrate;
b) coating the substrate with a resist;
c) etching the resist with an electron beam to produce a patterned substrate;
d) evaporating metal on the patterned substrate;
e) removing the resist to produce a metal nanostructure,
f) modifying the metal nanostructure with a ligand;
g) contacting the ligand-modified metal nanostructure with at least one cell;
h) allowing the cell to grow; and
i) optionally, differentiating the cell.

14. The method of claim 13, wherein the substrate comprises a metal.

15. The method of claim 13, wherein the resist comprises a positive resist.

16. The method of claim 13, wherein the patterned substrate has a pitch of about 10 nm to about 2,000 nm.

17. The method of claim 13, wherein the evaporated metal comprises gold.

18. The method of claim 13, wherein removing the resist comprises organic polymer.

19. The method of claim 13, wherein the metal nanostructure has a pitch of about 10 nm to about 2,000 nm.

20. The method of claim 13, wherein the metal nanostructure is circular, oval, square, rectangular, or star shaped.

21. The method of claim 13, wherein the ligand is a sulfur-containing compound, growth factor, cytokine, inhibitor of gene regulation, activator of gene regulation, growth hormone, peptide, or receptor.

22. The method of claim 13, wherein the at least one cell comprises a stem cell, embryonic stem cell, adult stem cell, adult mesenchymal, hematopoietic, neural, epithelial, skin stem cell, or progenitor cell.

23. The method of claim 13, wherein the cell is differentiated, and the differentiated at least one cell is a chondrogenic cell, an osteogenic cell, a neurogenic cell, a myogenic cell, adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocyte.

24. A method of cell culture, comprising:
a) providing a substrate;
b) coating the substrate with a resist;
c) etching the resist with an electron beam to produce a patterned substrate;
d) modifying the patterned substrate with a silane solution;
e) removing the resist to provide a silane-modified substrate region;
f) contacting the silane-modified substrate region with at least one cell;
g) allowing the cell to grow; and
g) optionally, differentiating the cell.

25. The method of claim 24, wherein the substrate comprises a polymer.

26. The method of claim 24, wherein the resist comprises a negative resist.

27. The method of claim 24, wherein the patterned substrate has a pitch of about 10 nm to about 2,000 nm.

28. The method of claim 24, wherein the silane solution comprises 0.5-2% (w/w) n-octadecyltrimethoxysilane (OTS)/toluene solution.

28. The method of claim 24, wherein removing the resist comprises etching.

30. The method of claim 24, wherein the silane-modified substrate region is circular, oval, square, rectangular, or star shaped.

31. The method of claim 24, wherein the silane-modified substrate region has a diameter of about 10 nm to about 2,000 nm.

32. The method of claim 24, wherein the at least one cell comprises a stem cell, embryonic stem cell, adult stem cell, adult mesenchymal, hematopoietic, neural, epithelial, skin stem cell, or progenitor cell.

33. The method of claim 24, wherein the differentiated at least one cell is a chondrogenic cell, an osteogenic cell, a neurogenic cell, a myogenic cell, adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocyte.

34. A method of cell culture, comprising:
a) providing a substrate;
b) coating the substrate with a metal;
c) coating the metal with a resist;
d) etching the resist with an electron beam to produce a patterned metal;
e) modifying the patterned metal with a silane solution;
f) removing the resist to provide a silane-modified metal region;
g) contacting the silane-modified metal with at least one cell;
h) allowing the cell to grow; and
i) optionally, differentiating the cell.

35. The method of claim 34, wherein the substrate comprises a polymer.

36. The method of claim 34, wherein the metal comprises gold.

37. The method of claim 34, wherein the resist comprises a positive resist.

38. The method of claim 34, wherein the patterned metal has a pitch of about 10 nm to about 2,000 nm.

39. The method of claim 34, wherein the silane solution comprises 0.5-2% (w/w) n-octadecyltrimethoxysilane (OTS)/toluene solution.

40. The method of claim 34, wherein removing the resist comprises etching.

41. The method of claim 34, wherein the silane-modified metal region is circular, oval, square, rectangular, or star shaped.

42. The method of claim 34, wherein the silane-modified metal region has a diameter of about 10 nm to about 2,000 nm.

43. The method of claim 34, wherein the at least one cell comprises a stem cell, embryonic stem cell, adult stem cell, adult mesenchymal, hematopoietic, neural, epithelial, skin stem cell, progenitor cell.

44. The method of claim 34, wherein the differentiated at least one cell is a chondrogenic cell, an osteogenic cell, a neurogenic cell, a myogenic cell, an adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocyte.

45. A method of cell culture, comprising:
a) providing a substrate;
b) coating the substrate with a resist;
c) fabricating a resist nanostructure with a nanoimprint stamp;
d) using reactive ion etching to expose regions of the substrate;
e) evaporating a metal on the resist nanostructures and exposed substrate regions;
f) removing the resist to provide a metal nanostructure;
g) modifying the metal nanostructure with a ligand;
h) contacting the modified nanostructure with at least one cell;
i) allowing the cell to grow; and
j) optionally, differentiating the cell.

46. The method of claim 45, wherein the substrate comprises a polymer.

47. The method of claim 45, wherein the resist comprises a negative resist.

48. The method of claim 45, wherein the resist nanostructure has a pitch of about 10 nm to about 2,000 nm.

49. The method of claim 45, wherein the metal comprises gold.

50. The method of claim 45, wherein the reactive ion etching is carried out under vacuum.

51. The method of claim 45, wherein the metal nanostructure is circular, oval, square, rectangular, or star shaped.

52. The method of claim 45, wherein the metal nanostructure has a diameter of about 10 nm to about 2,000 nm.

53. The method of claim 45, wherein the ligand is a sulfur-containing compound, growth factor, cytokine, inhibitor of gene regulation, activator of gene regulation, growth hormone, peptide, or receptor.

54. The method of claim 45, wherein the at least one cell comprises a stem cell, embryonic stem cell, adult stem cell, adult mesenchymal, hematopoietic, neural, epithelial, skin stem cell, or progenitor cell.

55. The method of claim 45, wherein the differentiated at least one cell is a chondrogenic cell, an osteogenic cell, a neurogenic cell, a myogenic cell, an adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocytes.

56. A method of cell culture, comprising:
a) providing a substrate;
b) coating the substrate with a resist;
c) fabricating a resist nanostructure with a nanoimprint stamp;
d) using reactive ion etching to expose regions of substrate;
e) exposing the exposed regions of substrate to a silane solution;
f) removing the resist to provide a silane nanostructure;
g) contacting the silane nanostructure with at least one cell;
h) allowing the cell to grow; and
i) optionally, differentiating the cell.

57. The method of claim 56, wherein the substrate comprises a polymer.

58. The method of claim 56, wherein the resist comprises a positive resist.

59. The method of claim 56, wherein the resist nanostructures have a pitch of about

60. The method of claim 56, wherein the silane solution comprises 0.5-2% (w/w) n-octadecyltrimethoxysilane (OTS)/toluene solution.

61. The method of claim 56, wherein the reactive ion etching comprises use of vacuum conditions.

62. The method of claim 56, wherein the silane nanostructure is circular, oval, square, rectangular, or star shaped.

63. The method of claim 56, wherein the silane nanostructure has a diameter of about 10 nm to about 2,000 nm.

64. The method of claim 56, wherein the ligand is a sulfur-containing compound, growth factor, cytokine, inhibitor of gene regulation, activator of gene regulation, growth hormone, peptide, or receptor.

65. The method of claim 56, wherein the at least one cell comprises a stem cell, embryonic stem cell, adult stem cell, progenitor cell, adult mesenchymal, hematopoietic, neural, epithelial, or skin stem cell.

66. The method of claim 56, wherein the differentiated cell is a chondrogenic cell, an osteogenic cell, a neurogenic cell, a myogenic cell, an adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocyte.

67. A method of cell culture, comprising:
a) providing a substrate;
b) coating the substrate with a metal;
c) coating the metal with a resist;
c) fabricating a resist nanostructure with a nanoimprint stamp;
d) using reactive ion etching to expose regions of the metal;
e) modifying the exposed metal regions with a ligand:
f) removing the resist to provide a ligand nanostructure;
g) contacting the ligand nanostructure with at least one cell;
h) allowing the cell to grow; and
i) optionally, differentiating the cell.

68. The method of claim 67, wherein the substrate comprises a polymer.

69. The method of claim 67, wherein the metal comprises gold.

70. The method of claim 67, wherein the resist comprises a positive resist.

71. The method of claim 67, wherein the resist nanostructure has a pitch of about 10 nm to about 2,000 nm.

72. The method of claim 67, wherein the resist is a negative resist.

73. The method of claim 67, wherein the reactive ion etching comprises use of vacuum conditions.

74. The method of claim 67, wherein the exposed regions of metal are circular, oval, square, rectangular, or star shaped.

75. The method of claim 67, wherein the exposed regions of metal have a diameter of about 10 nm to about 2,000 nm.

76. The method of claim 67, wherein the ligand is a sulfur-containing compound, growth factor, cytokine, inhibitor of gene regulation, activator of gene regulation, growth hormone, peptide, or receptor.

77. The method of claim 67, wherein the at least one cell comprises a stem cell, embryonic stem cell, adult stem cell, adult mesenchymal, hematopoietic, neural, epithelial, skin stem cell, or progenitor cell.

78. The method of claim 67, wherein the differentiated cell is a chondrogenic cell, an osteogenic cell, a neurogenic cell, a myogenic cell, an adipogenic cell, red blood cell, B lymphocyte, T lymphocyte, natural killer cell, neutrophil, basophil, eosinophil, monocyte, macrophage, platelet, neuron, oligodendrocyte, astrocyte, absorptive cell, globlet cell, paneth cell, enteroendocrine cell, or keratinocyte.

79. A method of cell culture, comprising:
a) providing a ligand-modified nanostructure prepared by a direct write nanolithography technique, wherein the direct write nanolithography technique is dip pen nanolithography, microcontact printing, nanografting, or nanopen reader writer nanolithography
b) contacting the ligand-modified nanostructure with at least one cell;
c) allowing the at least one cell to grow; and
d) optionally, differentiating the at least one cell.
